(19) United States
(12) Patent Application Publication
(10) Pub. No.: US 2012/0029306 A1
(43) Pub. Date: Feb. 2, 2012

(54) VITAL-SIGNS MONITOR WITH ENCAPSULATION ARRANGEMENT

(75) Inventors: Pierre Paquet, Quebec (CA); Darren Macfarlane, York (GB)

(73) Assignee: Carefusion 303, Inc., San Diego, CA (US)

(21) Appl. No.: 12/844,766

(22) Filed: Jul. 27, 2010

(51) Int. Cl. A61B 5/00

(52) U.S. Cl. 600/301

(57) ABSTRACT

A vital-signs monitoring device is disclosed. The vital-signs monitor includes a sensor that measures a physiological parameter of a patient, a circuit assembly containing vital-signs monitoring circuitry that analyzes the sensor measurements to generate vital sign signals, and a housing. The housing is designed to be worn by a patient and encapsulates the circuit assembly such that moisture and particulate matter is prevented from reaching the circuit assembly through the housing.

### CROSS-REFERENCES TO RELATED APPLICATIONS

The following applications disclose certain common subject matter with the present application: A Vital-Signs Monitor with Spaced Electrodes, docket number 080624-0623; A Vital-Signs Patch Having a Strain Relief, docket number 080624-0624; A Temperature Probe Suitable for Axillary Reading, docket number 080624-0625; System and Method for Monitoring Body Temperature of a Person, docket number 080624-0626; A System and Method for Storing and Forwarding Data from a Vital-Signs Monitor, docket number 080624-0627; System and Method for Saving Battery Power in a Vital Signs Monitor, docket number 080624-0628; A System and Method for Conserving Battery Power in a Patient Monitoring System, docket number 080624-0629; A System and Method for Saving Battery Power in a Patient Monitoring System, docket number 080624-0630; A System And Method for Tracking Vital-Signs Monitor Patches, Docket Number 080624-0631; A System And Method for Reducing False Alarms Associated with Vital-Signs Monitoring, docket number 080624-0632; A System And Method for Location Tracking of Patients in a Vital-Signs Monitoring System, docket number 080624-0633; A System And Method for Reducing False Alarms Based on Motion and Location Sensing, docket number 080624-0634; all of the listed applications filed on ______.

### FIELD

The present disclosure generally relates to systems and methods of physiological monitoring, and, in particular, relates to monitoring of vital signs of patients.

### DESCRIPTION OF THE RELATED ART

Some of the most basic indicators of a person's health are those physiological measurements that reflect basic body functions and are commonly referred to as a person's “vital signs.” The four measurements commonly considered to be vital signs are body temperature, pulse rate, blood pressure, and respiratory rate. Some clinicians consider oxygen saturation (S₀₂) to be a “fifth vital sign” particularly for pediatric or geriatric cases. Some or all of these measurements may be performed routinely upon a patient when they arrive at a healthcare facility, whether it is a routine visit to their doctor or arrival at an Emergency Room (ER).

Vital signs are frequently taken by a nurse using basic tools including a thermometer to measure body temperature, a sphygmomanometer to measure blood pressure, and a watch to count the number of breaths or the number of heart beats in a defined period of time which is then converted to a “per minute” rate. If a patient's pulse is weak, it may not be possible to detect a pulse by hand and the nurse may use a stethoscope to amplify the sound of the patient's heart beat so that she can count the beats. Oxygen saturation of the blood is most easily measured with a pulse oximeter.

When a patient is admitted to a hospital, it is common for vital signs to be measured and recorded at regular intervals during the patient's stay to monitor their condition. A typical interval is 4 hours, which leads to the undesirable requirement for a nurse to awaken a patient in the middle of the night to take vital sign measurements.

When a patient is admitted to an ER, it is common for a nurse to do a “triage” assessment of the patient's condition that will determine how quickly the patient receives treatment. During busy times in an ER, a patient who does not appear to have a life-threatening injury may wait for hours until more-serious cases have been treated. While the patient may be reassessed at intervals while awaiting treatment, the patient may not be under observation between these reassessments.

Measuring certain vital signs is normally intrusive at best and difficult to do on a continuous basis. Measurement of body temperature, for example, is commonly done by placing an oral thermometer under the tongue or placing an infrared thermometer in the ear canal such that the tympanic membrane, which shared blood circulation with the brain, is in the sensor's field of view. Another method of taking a body temperature is by placing a thermometer under the arm, referred to as an “axillary” measurement as axilla is the Latin word for armpit. Skin temperature can be measured using a stick-on strip that may contain panels that change color to indicate the temperature of the skin below the strip.

Measurement of respiration is easy for a nurse to do, but relatively complicated for equipment to achieve. A method of automatically measuring respiration is to encircle the upper torso with a flexible band that can detect the physical expansion of the rib cage when a patient inhales. An alternate technique is to measure a high-frequency electrical impedance between two electrodes placed on the torso and detect the change in impedance created when the lungs fill with air. The electrodes are typically placed on opposite sides of one or both lungs, resulting in placement on the front and back or on the left and right sides of the torso, commonly done with adhesive electrodes connected by wires or by using a torso band with multiple electrodes in the strap.

Measurement of pulse is also relatively easy for a nurse to do and intrusive for equipment to achieve. A common automatic method of measuring a pulse is to use an electrocardiograph (ECG or EKG) to detect the electrical activity of the heart. An EKG machine may use 12 electrodes placed at defined points on the body to detect various signals associated with the heart function. Another common piece of equipment is simply called a “heart rate monitor.” Widely sold for use in exercise and training, heart rate monitors commonly consist of a torso band, in which are embedded two electrodes held against the skin and a small electronics package. Such heart rate monitors can communicate wirelessly to other equipment such as a small device that is worn like a wristwatch and that can transfer data wirelessly to a PC.

Nurses are expected to provide complete care to an assigned number of patients. The workload of a typical nurse is increasing, driven by a combination of a continuing shortage of nurses, an increase in the number of formal procedures that must be followed, and an expectation of increased documentation. Replacing the manual measurement and logging of vital signs with a system that measures and records vital signs would enable a nurse to spend more time on other activities and avoid the potential for error that is inherent in any manual procedure.

### SUMMARY

For some or all of the reasons listed above, there is a need to be able to continuously monitor patients in different settings. In addition, it is desirable for this monitoring to be done with limited interference with a patient's mobility or interfering with their other activities.

Embodiments of the patient monitoring system disclosed herein measure certain vital signs of a patient, which include respiratory rate, pulse rate, blood pressure, body temperature, and, in some cases, oxygen saturation (S_{O2}), on a regular basis and compare these measurements to defined limits.

In certain aspects of the present disclosure, a vital-signs monitor patch is disclosed. The monitor patch includes at least one sensor, a circuit assembly, and a housing configured to be worn by a patient. The housing encapsulates the circuit assembly prevent moisture and particulate matter from reaching the circuit assembly.

In certain aspects of the present disclosure, a vital-signs patch is disclosed. The monitor patch includes at least one sensor, a circuit assembly, and a housing configured to be worn by a patient. The housing includes first and second layers that are sealed together to form a hermetic encapsulation of the circuit assembly.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a diagram illustrating an exemplary embodiment of a patient monitoring system according to certain aspects of the present disclosure.

FIG. 2A is a perspective view of the vital-signs monitor patch of FIG. 1 according to certain aspects of the present disclosure.

FIG. 2B is a cross-section of the vital-signs monitor patch of FIG. 1 according to certain aspects of the present disclosure.

FIG. 2C is a functional block diagram illustrating exemplary electronic and sensor components of the vital-signs monitor patch of FIG. 1 according to certain aspects of the present disclosure.

FIG. 3 is a cross-section of an exemplary embodiment of the vital-signs patch according to certain aspects of the present disclosure.

FIGS. 4A-4E show perspective and cross-section views of the removable power isolation strip according to certain embodiments of the present disclosure.

FIGS. 5A and 5B show a perspective view and a schematic view of an alternate activation configuration according to certain embodiments of the present disclosure.

### DETAILED DESCRIPTION

Periodic monitoring of patients in a hospital is desirable at least to ensure that patients do not suffer an un-noticed sudden deterioration in their condition or a secondary injury during their stay in the hospital. It is impractical to provide continuous monitoring by a clinician and cumbersome to connect sensors to a patient, which are then connected to a fixed monitoring instrument by wires. Furthermore, systems that sound an alarm when the measured value exceeds a threshold value may sound alarms so often and in situations that are not truly serious that such alarms are ignored by clinicians.

Measuring vital signs is difficult to do on a continuous basis. Accurate measurement of cardiac pulse, for example, can be done using an electrocardiograph (ECG or EKG) to detect the electrical activity of the heart. An EKG machine may use up to 12 electrodes placed at various points on the body to detect various signals associated with the cardiac function. Another common piece of equipment is termed a “heart rate monitor.” Widely sold for use in exercise and physical training, heart rate monitors may comprise a torso band in which are embedded two electrodes held against the skin and a small electronics package. Such heart rate monitors can communicate wirelessly to other equipment such as a small device that is worn like a wristwatch and that can transfer data wirelessly to a personal computer (PC).

Monitoring of patients that is referred to as “continuous” is frequently periodic, in that measurements are taken at intervals. In many cases, the process to make a single measurement takes a certain amount of time, such that even back-to-back measurements produce values at an interval equal to the time that it takes to make the measurement. For the purpose of vital sign measurement, a sequence of repeated measurements can be considered to be “continuous” when the vital sign is not likely to change an amount that is of clinical significance within the interval between measurements. For example, a measurement of blood pressure every 10 minutes may be considered “continuous” if it is considered unlikely that a patient's blood pressure can change by a clinically significant amount within 10 minutes. The interval appropriate for measurements to be considered continuous may depend on a variety of factors including the type of injury or treatment and the patient's medical history. Compared to intervals of 4-8 hours for manual vital sign measurement in a hospital, measurement intervals of 30 minutes to several hours may still be considered “continuous.”

Certain exemplary embodiments of the present disclosure include a system that comprises a vital-signs monitor patch that is attached to the patient, and a bridge that communicates with monitor patches and links them to a central server that processes the data, where the server can send data and alarms to a hospital system according to algorithms and protocols defined by the hospital.

The construction of the vital-signs monitor patch is described according to certain aspects of the present disclosure. As the patch may be worn continuously for a period of time that may be several days, as is described in the following disclosure, it is desirable to encapsulate the components of the patch such that the patient can bathe or shower and engage in their normal activities without degradation of the patch function. An exemplary configuration of the construction of the patch to provide a hermetically sealed enclosure about the electronics is disclosed.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

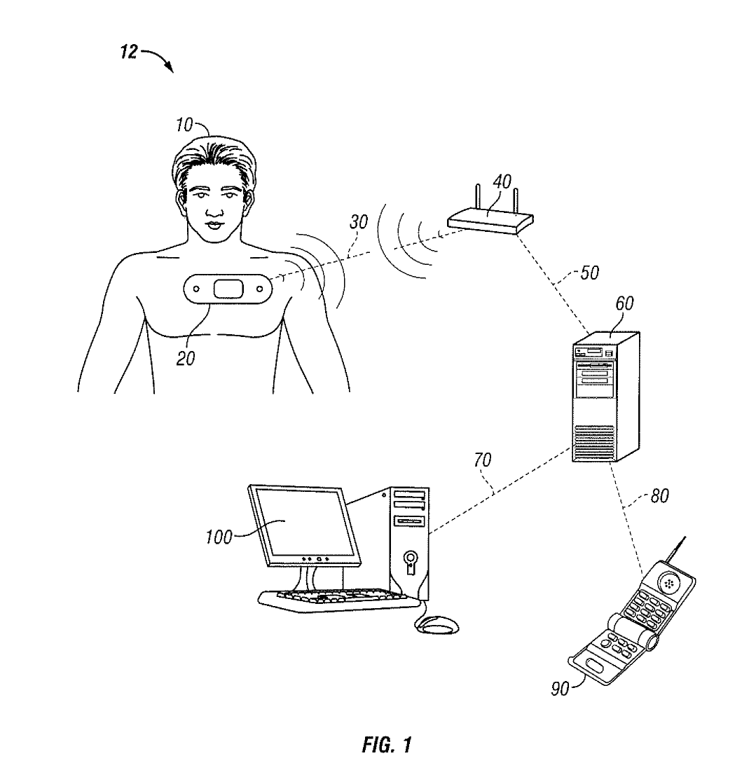

FIG. 1 discloses a vital sign monitoring system according to certain embodiments of the present disclosure. The vital sign monitoring system 12 includes vital-signs monitor patch 20, bridge 40, and surveillance server 60 that can send messages or interact with peripheral devices exemplified by mobile device 90 and workstation 100.

Monitor patch 20 resembles a large adhesive bandage and is applied to a patient 10 when in use. It is preferable to apply the monitor patch 20 to the upper chest of the patient 10 although other locations may be appropriate in some circumstances. Monitor patch 20 incorporates one or more electrodes (not shown) that are in contact with the skin of patient 10 to measure vital signs such as cardiac pulse rate and respiration rate. Monitor patch 20 also may include other sensors such as an accelerometer, temperature sensor, or oxygen saturation sensor to measure other characteristics associated with the patient. These other sensors may be internal to the monitor patch 20 or external sensors that are operably connected to the monitor patch 20 via a cable or wireless connection. Monitor patch 20 also includes a wireless transmitter that can both transmit and receive signals. This transmitter is preferably a short-range, low-power radio frequency (RF) device operating in one of the unlicensed radio bands. One band in the United States (US) is, for example, centered at 915 MHz and designated for industrial, scientific and medical (ISM) purposes. An example of an equivalent band in the European Union (EU) is centered at 868 MHz. Other frequencies of operation may be possible dependent upon the International Telecommunication Union (ITU), local regulations and interference from other wireless devices.

Surveillance server 60 may be a standard computer server connected to the hospital communication network and preferably located in the hospital data center or computer room, although other locations may be employed. The server 60 stores and processes signals related to the operation of the patient monitoring system 12 disclosed herein including the association of individual monitor patches 20 with patients 10 and measurement signals received from multiple monitor patches 20. Hence, although only a single patient 10 and monitor patch 20 are depicted in FIG. 1, the server 60 is able to monitor the monitor patches 20 for multiple patients 10.

Bridge 40 is a device that connects, or “bridges”, between monitor patch 20 and server 60. Bridge 40 communicates with monitor patch 20 over communication link 30 operating, in these exemplary embodiments, at approximately 915 MHz and at a power level that enables communication link 30 to function up to a distance of approximately 10 meters. It is preferable to place a bridge 40 in each room and at regular intervals along hallways of the healthcare facility where it is desired to provide the ability to communicate with monitor patches 20. Bridge 40 also is able to communicate with server 60 over network link 50 using any of a variety of computer communication systems including hardwired and wireless Ethernet using protocols such as 802.11a/b/g or 802.3af. As the communication protocols of communication link 30 and network link 50 may be very different, bridge 40 provides data buffering and protocol conversion to enable bidirectional signal transmission between monitor patch 20 and server 60.

While the embodiments illustrated by FIG. 1 employ a bridge 20 to provide communication link between the monitor patch 20 and the server 60, in certain alternative embodiments, the monitor patch 20 may engage in direct wireless communication with the server 60. In such alternative embodiments, the server 60 itself or a wireless modem connected to the server 60 may include a wireless communication system to receive data from the monitor patch 20.

In use, a monitor patch 20 is applied to a patient 10 by a clinician when it is desirable to continuously monitor basic vital signs of patient 10 while patient 10 is, in this embodiment, in a hospital. Monitor patch 2Q is intended to remain attached to patient 10 for an extended period of time, for example, up to 5 days in certain embodiments, limited by the battery life of monitor patch 20. In some embodiments, monitor patch 20 is disposable when removed from patient 10.

Server 60 executes analytical protocols on the measurement data that it receives from monitor patch 20 and provides this information to clinicians through external workstations 100, preferably personal computers (PCs), laptops, or smart phones, over the hospital network 70. Server 60 may also send messages to mobile devices 90, such as cell phones or pagers, over a mobile device link 80 if a measurement signal exceeds specified parameters. Mobile device link 80 may include the hospital network 70 and internal or external wireless communication systems that are capable of sending messages that can be received by mobile devices 90.

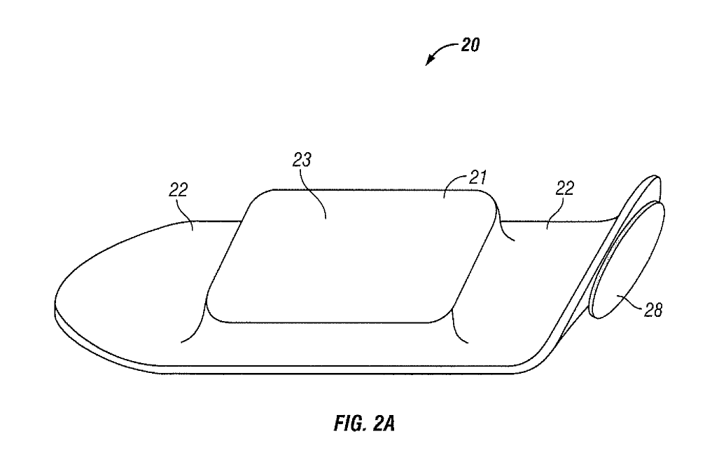

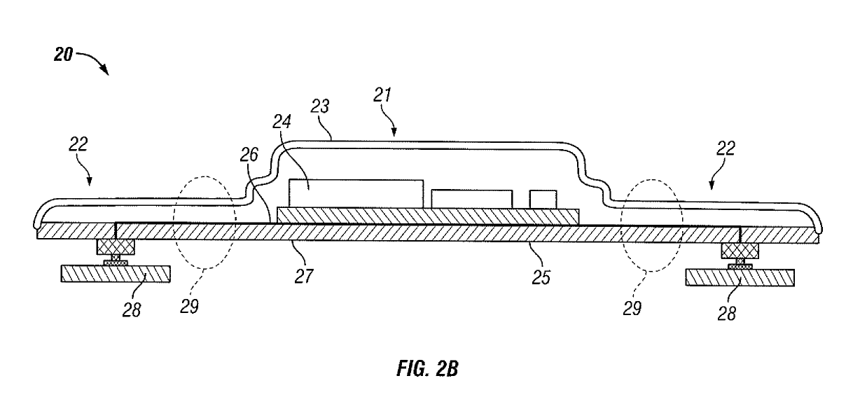

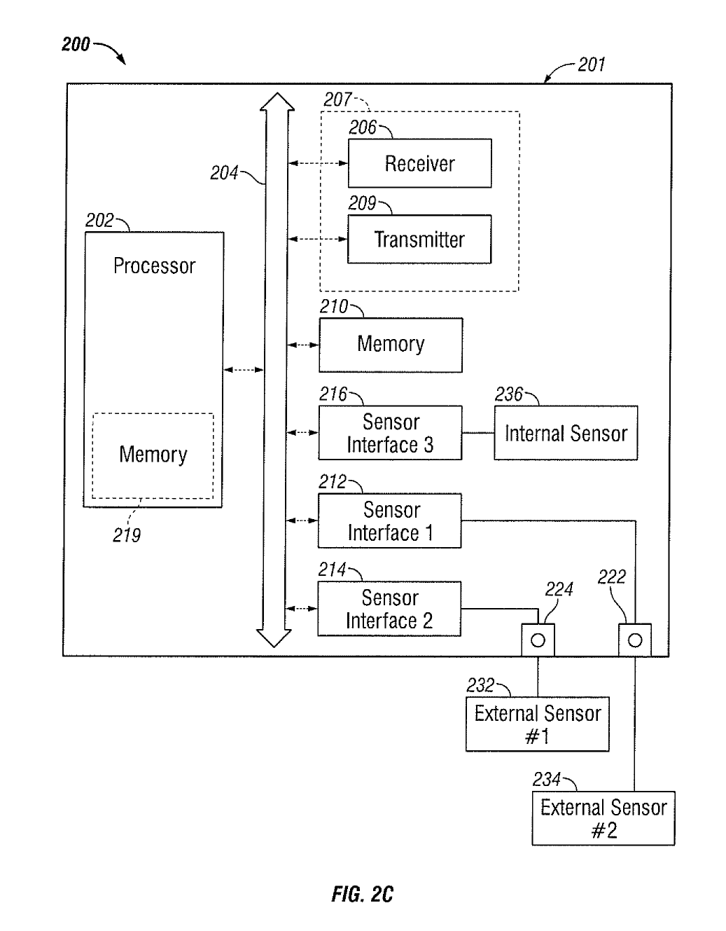

FIG. 2A is a perspective view of the vital-signs monitor patch 20 shown in FIG. 1 according to certain aspects of the present disclosure. In the illustrated embodiment, the monitor patch 20 includes component carrier 23 comprising a central segment 21 and side segments 22 on opposing sides of the central segment 21. In certain embodiments, the central segment 21 is substantially rigid and includes a circuit assembly (24, FIG. 2B) having electronic components and battery mounted to a rigid printed circuit board (PCB). The side segments 22 are flexible and include a flexible conductive circuit (26, FIG. 2B) that connect the circuit assembly 24 to electrodes 28 disposed at each end of the monitor patch 20, with side segment 22 on the right shown as being bent upwards for purposes of illustration to make one of the electrodes 28 visible in this view.

FIG. 2B is a cross-sectional view of the vital-signs patch 20 shown in FIGS. 1 and 2A according to certain aspects of the present disclosure. The circuit assembly 24 and flexible conductive circuit 26 described above can be seen herein. The flexible conductive circuit 26 operably connects the circuit assembly 24 to the electrodes 28. Top and bottom layers 23 and 27 form a housing 25 that encapsulate circuit assembly 28 to provide a water and particulate barrier as well as mechanical protection. Top and bottom layers 23 and 27 may comprise one or more materials or layers of material. There are sealing areas on layers 23 and 27 that encircles circuit assembly 28 and is visible in the cross-section view of FIG. 2B as areas 29. Layers 23 and 27 may be sealed to each other in this area to form a substantially hermetic seal. Within the context of certain aspects of the present disclosure, the term ‘hermetic’ implies that the rate of transmission of moisture through the seal is substantially the same as through the material of the layers that are sealed to each other, and further implies that the size of particulates that can pass through the seal are below the size that can have a significant effect on circuit assembly 24. Flexible conductive circuit 26 passes through portions of sealing areas 29 and the seal between layers 23 and 27 is maintained by sealing of layers 23 and 27 to flexible conductive circuit 26. The layers 23 and 27 are thin and flexible, as is the flexible conductive circuit 26, allowing the side segment 22 of the monitor patch 20 between the electrodes 28 and the circuit assembly 24 to bend as shown in FIG. 2A.

FIG. 2C is a functional block diagram 200 illustrating exemplary electronic and sensor components of the monitor patch 20 of FIG. 1 according to certain aspects of the present disclosure. The block diagram 200 shows a processing and sensor interface module 201 and external sensors 232, 234 connected to the module 201. In the illustrated example, the module 201 includes a processor 202, a wireless transceiver 207 having a receiver 206 and a transmitter 209, a memory 210, a first sensor interface 212, a second sensor interface 214, a third sensor interface 216, and an internal sensor 236 connected to the third sensor interface 216. The first and second sensor interfaces 212 and 214 are connected to the first and second external sensors 232, 234 via first and second connection ports 222, 224, respectively. In certain embodiments, some or all of the aforementioned components of the module 201 and other components are mounted on a PCB.

Each of the sensor interfaces 212, 214, 216 can include one or more electronic components that are configured to generate an excitation signal or provide DC power for the sensor that the interface is connected to and/or to condition and digitize a sensor signal from the sensor. For example, the sensor interface can include a signal generator for generating an excitation signal or a voltage regulator for providing power to the sensor. The sensor interface can further include an amplifier for amplifying a sensor signal from the sensor and an analog-to-digital converter for digitizing the amplified sensor signal. The sensor interface can further include a filter (e.g., a low-pass or bandpass filter) for filtering out spurious noises (e.g., a 60 Hz noise pickup).

The processor 202 is configured to send and receive data (e.g., digitized signal or control data) to and from the sensor interfaces 212, 214, 216 via a bus 204, which can be one or more wire traces on the PCB. Although a bus communication topology is used in this embodiment, some or all communication between discrete components can also be implemented as direct links without departing from the scope of the present disclosure. For example, the processor 202 may send data representative of an excitation signal to the sensor excitation signal generator inside the sensor interface and receive data representative of the sensor signal from the sensor interface, over either a bus or direct data links between processor 202 and each of sensor interface 212, 214, and 216.

The processor 202 is also capable of communication with the receiver 206 and the transmitter 209 of the wireless transceiver 207 via the bus 204. For example, the processor 202 using the transmitter and receiver 209, 206 can transmit and receive data to and from the bridge 40. In certain embodiments, the transmitter 209 includes one or more of a RF signal generator (e.g., an oscillator), a modulator (a mixer), and a transmitting antenna; and the receiver 206 includes a demodulator (a mixer) and a receiving antenna which may or may not be the same as the transmitting antenna. In some embodiments, the transmitter 209 may include a digital-to-analog converter configured to receive data from the processor 202 and to generate a base signal; and/or the receiver 206 may include an analog-to-digital converter configured to digitize a demodulated base signal and output a stream of digitized data to the processor 202. In other embodiments, the radio may comprise a direct sequence radio, a software-defined radio, or an impulse spread spectrum radio.

The processor 202 may include a general-purpose processor or a specific-purpose processor for executing instructions and may further include a memory 219, such as a volatile or non-volatile memory, for storing data and/or instructions for software programs. The instructions, which may be stored in a memory 219 and/or 210, may be executed by the processor 202 to control and manage the wireless transceiver 207, the sensor interfaces 212, 214, 216, as well as provide other communication and processing functions.

The processor 202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information, such as program instructions, data representative of sensor readings, preset alarm conditions, threshold limits, may be stored in a computer or processor readable medium such as a memory internal to the processor 202 (e.g., the memory 219) or a memory external to the processor 202 (e.g., the memory 210), such as a Random Access Memory (RAM), a flash memory, a Read Only Memory (ROM), a Programmable Read-Only Memory (PROM), an Erasable PROM (EPROM), registers, a hard disk, a removable disk, or any other suitable storage device.

In certain embodiments, the internal sensor 236 can be one or more sensors configured to measure certain properties of the processing and sensor interface module 201, such as a board temperature sensor thermally coupled to a PCB. In other embodiments, the internal sensor 236 can be one or more sensors configured to measure certain properties of the patient 10, such as a motion sensor (e.g., an accelerometer) for measuring the patient's motion or position with respect to gravity.

The external sensors 232, 234 can include sensors and sensing arrangements that are configured to produce a signal representative of one or more vital signs of the patient to which the monitor patch 20 is attached. For example, the first external sensor 232 can be a set of sensing electrodes that are affixed to an exterior surface of the monitor patch 20 and configured to be in contact with the patient for measuring the patient's respiratory rate, and the second external sensor 234 can include a temperature sensing element (e.g., a thermocouple or a thermistor or resistive thermal device (RTD)) affixed, either directly or via an interposing layer, to skin of the patient 10 for measuring the patient's body temperature. In other embodiments, one or more of the external sensors 232, 234 or one or more additional external sensors can measure other vital signs of the patient, such as blood pressure, pulse rate, or oxygen saturation.

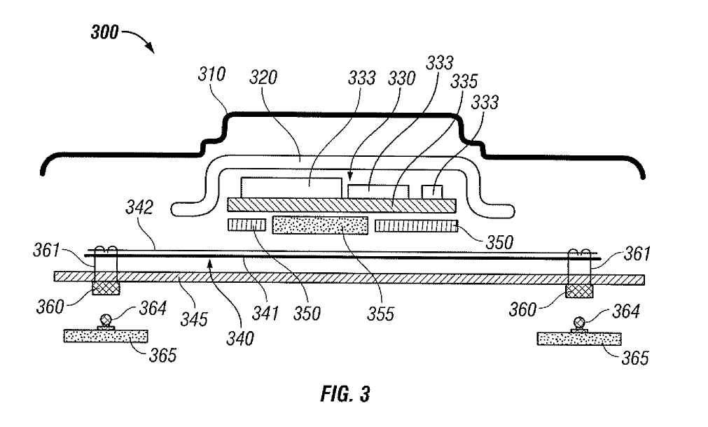

FIG. 3 is an exploded cross-section view 300 of an exemplary embodiment of the vital-signs patch similar to that of FIG. 2B with additional components and details of construction shown. Circuit assembly 330 comprises electronic circuit components 333 mounted, in this configuration, on a rigid printed circuit board (PCB) 335. There are two electrodes 365 located on opposite sides of circuit assembly 330. Flexible conductive circuit 340, comprising a flexible substrate 341 and conductive strips 342 located on at least one surface of the substrate 341, connects electrodes 365 to circuit assembly 330. Conductive strips 342 may be applied to the substrate 341 via a number of processes known to those skilled in the art including plating of copper onto a substrate, silk-screening of conductive ink onto a compatible plastic sheet, and screen printing with conductive ink on a substrate.

Electrodes 365 removably connect to receptacles 360 through snap fittings 364 that mate with receptacles 360. Electrical contact between receptacles 360 and conductive strips 342 is established through conductors 361 that protrude from receptacles 360 and, in this configuration, penetrate both bottom protective film 345 and substrate 341 and are crimped back into electrical contact with conductive strips 342. The electrical connection between the conductive strips 342 on flexible circuit 340 and the circuits of the circuit assembly 330 is made through an conductive adhesive 355 which covers an area (not referenced) of PCB 335 that, in this embodiment, has exposed circuit traces (not shown). The circuit traces of PCB 335 and the conductive strips 342 of the flexible circuit 340 are laid out in such a manner that exposed conductive elements of the two overlap only in locations where electrical connection is desired. A nonconductive layer may be applied to one of or both the PCB 335 and flexible circuit 340 to prevent electrical contact in unintended areas. In some embodiments, conductive adhesive 355 may be an isotropically conductive adhesive. In some other embodiments, conductive adhesive 355 may be an anisotropically conductive adhesive. Conductive adhesive should ideally be held in compression to maintain the electrically conductive path through the adhesive.

In the embodiment of FIG. 3, segments of high-strength adhesive 350 are placed substantially adjacent to the conductive adhesive 355. In other embodiments, there may be only a single segment of high-strength adhesive 350. In some embodiments, the layers of high-strength adhesive 350 may be thinner than the layer of conductive adhesive 355 so that after flexible circuit 340 is pressed against both adhesive segments 350 and 355 during assembly, the high strength adhesive 350 will be in tension while the conductive adhesive 355 will be in compression.

The bottom protective film 345 is chosen for a number of attributes. Film 345 may comprise more than one layer or coatings, and the exterior surface layer is intended to be biocompatible and preferably hypoallergenic. Film 345 should have a low permeability to moisture to protect the internal electronics even if exposed to water, for example, while showering. The film 345 should be formable in thin films and flexible to conform to the human body. At the same time, it is desirable for the material to feel soft against the skin. In certain embodiments, film 345 comprises a foam material.

The top protective film 310 is chosen for biocompatibility, flexibility, low moisture permeability and easy cleanability. In this case, a smooth exterior surface is desirable. A material such as a waterproof polyurethane film is suitable. Top protective film may comprise one or more layers of material. It is preferred to seal the top protective film 310 to substrate 341 around the perimeter of the circuit assembly 330. This may be accomplished through heat-sealing if the materials of layers 310 and 345 are compatible. Alternately, layers 310 and 345 may be bonded by any suitable standard process including ultrasonic welding, solvent bonding, adhesive cured by heat, moisture, or ultraviolet light, or use of an intermediate layer (not shown) that adheres to both layers 310 and 345. In areas where the flexible conductive circuit 340 crosses the sealing perimeter, layers 310 and 345 are sealed to the flexible conductive circuit 340.

A cushioning layer 320 may be optionally added to provide both mechanical protection of the electronics 333 on circuit assembly 330 and additional comfort for the patient and clinician by masking sharp edges and protruding components. It is preferred to use a foam sheet of a size to cover the top and edges of the circuit assembly 330 but not protrude much beyond this to avoid interference with the bonding of layer 310 and substrate 341. It is also preferred to heat the cushioning layer 320 at the time of assembly such that the underside of the layer 320 conforms to the components of circuit assembly 330. While it is not required for cushioning layer 320 to adhere to circuit assembly 330, it is not detrimental and may ease assembly.

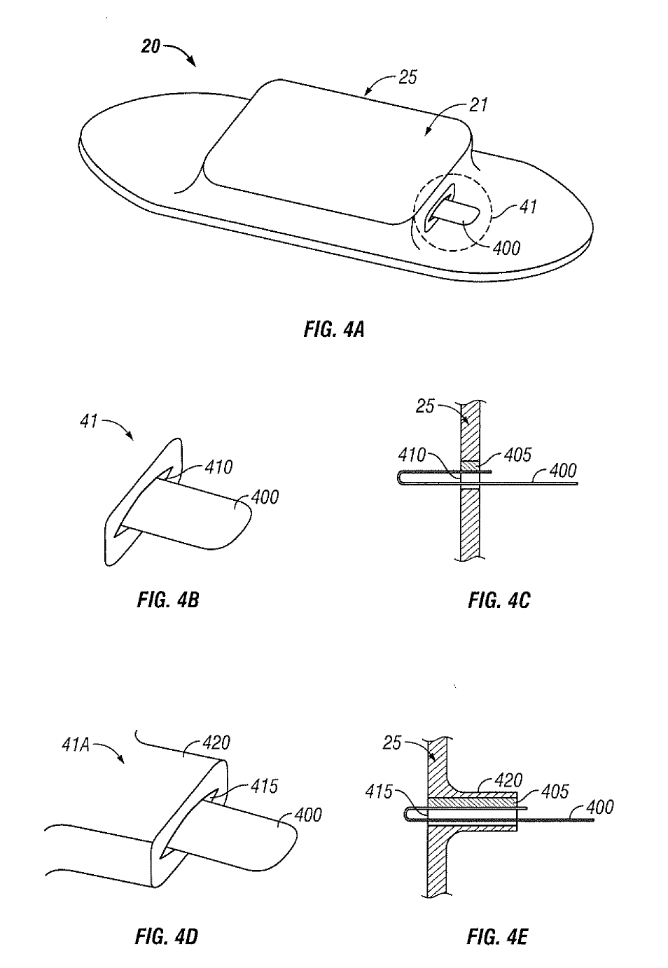

FIG. 4A is a perspective view of patch 20 showing an optional removable power strip 400 protruding through housing 25 on one side of central segment 21 which contains the circuit assembly (not shown). In other embodiments, this protrusion may be on any surface of the housing 25. This power isolation strip comprises an insulating material and is placed, at its interior end, between two elements of the power circuit. This can be accomplished in any of a number of methods know to those skilled in the art. Certain embodiments place the interior end of strip 400 between one surface of the battery and its spring-loaded mating contact (not shown). The power isolation strip of this type prevents the battery from discharging while in storage while simultaneously improving the reliability of the patch by eliminating a switch. In this embodiment, patch 20 is activated by removal of power strip 400. It is desirable, however, to seal the opening through which the power strip 400 protrudes after power isolation strip 400 is removed to complete the hermetic seal of housing 25.

FIGS. 4B and 4D disclose enlarged views of area 41 around the protrusion 400. In FIG. 4B, there is a slit 410 in housing 25 through which power isolation strip 400 protrudes. A cross-section of area 41 is shown in FIG. 4C. One surface of slit 410 is coated with adhesive 405. Strip 400 may be folded as shown in FIG. 4C to provide a lower release force when strip 400 is withdrawn from slit 410. The surface of strip 400 may be coated with an anti-stick coating to reduce the adhesion of adhesive 405. When strip 400 is removed from housing 25, the two sides of slit 410 will come together and adhesive 405 will stick to the other side, sealing this opening to prevent ingress of moisture or particulates.

An alternate embodiment is disclosed in FIG. 4D, where a tube 420 has been formed in the housing 25 with an inner surface 415 that is a slit penetrating the length of tube 420. In its relaxed state, the two surfaces of inner surface 415 are in contact with each other. FIG. 4E shows a cross-section of this embodiment, where it can be seen that the inner surface 415 of tube 420 is longer than the slit 410 of FIG. 4C. One surface of inner surface 415 is coated with an adhesive 405 of the same type as in the embodiment of FIGS. 4B and 4C. When power isolation strip 400 is removed from the housing 25 of this embodiment, the two surfaces of inner surface 415 will come into contact and the adhesive 405 will seal the surfaces to each other. The increased length of surface 415, compared to slit 410, may increase the quality and reliability of the seal.

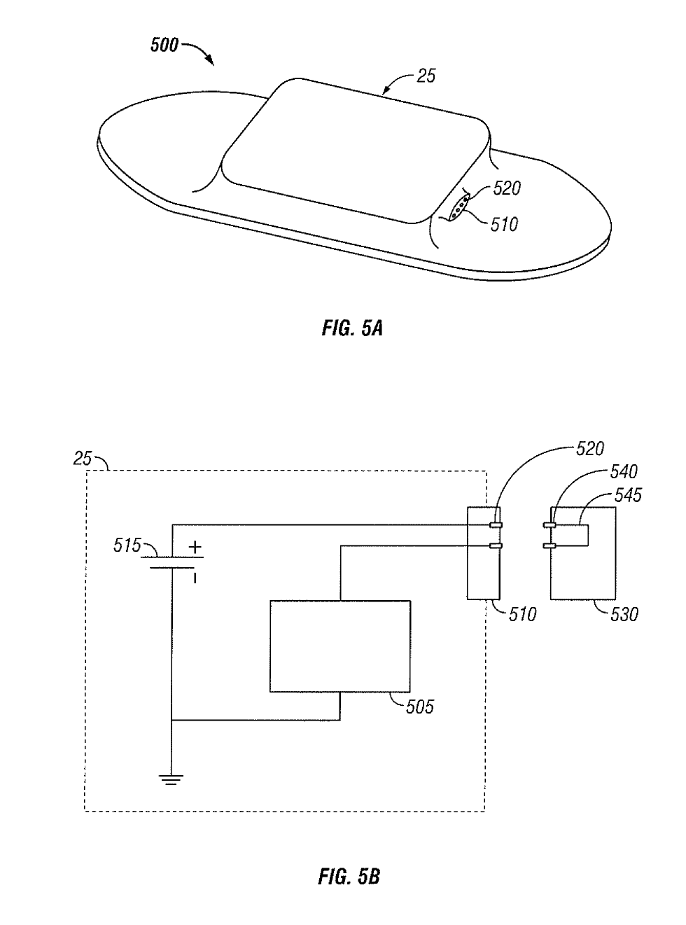

FIGS. 5A and 513 show a perspective view and a schematic view of an alternate activation configuration according to certain embodiments of the present disclosure. In this embodiment, vital-signs patch 500 comprises an external connector 510 having a plurality of contacts 520. In the example schematic of FIG. 5B, two of the contacts 520 are part of the circuit that connects the battery 515 to the rest of the circuit assembly 505. Plug 530 is constructed to mate with connector 510 and has two mating contacts 540 that are connected by jumper 545. When plug 530 is connected to connector 510, jumper 545 completes the power circuit from the battery 515 to the rest of the circuit assembly 505 and the vital-signs patch 500 starts to function. Other circuit designs for isolating the battery 515 from the rest of the circuit assembly 505 until a connector is mated to connector 510 include switches, transistors, microcomputers, and other devices that change state upon grounding of or application of power to a connection of that device. The plug 530 may, in certain embodiments, include other circuits and contacts to provide other functions to the vital-signs patch 500, such as connection of an external sensor, and the activation feature may be associated with the signals or grounding of those other functions.

It can be seen that the disclosed embodiments of the vital-signs monitor patch provide a mobile solution to monitoring the vital signs of a patient. The design of the vital-signs monitor patch frees nurses, or other caregivers, from the task of repetitively measuring the vital signs of their patients, allowing the caregivers to spend more time on other duties. The ability to continuously monitor a patient's vital signs using a monitor patch, together with the rest of the patient monitoring system, increases the ability of the nurse to respond quickly to a sudden change in a patient's condition, resulting in improved care for the patient. The hermetic seal provided by the patch housing protects the patch electronics from moisture and particulates while the device is worn by the patient, enabling the patient to shower or bathe while in the hospital and reduces limitations on activities of the patient that might otherwise be imposed to avoid damage or contamination of the patch electronics.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A vital-signs monitor patch comprising:
at least one sensor configured to measure a physiological parameter of a patient;
a circuit assembly containing vital-signs monitoring circuitry that analyzes the sensor measurements to generate vital sign signals; and
a housing that encapsulates the circuit assembly, configured for wearing by a patient, wherein the housing encapsulates the circuit assembly such that moisture and particulate matter is prevented from reaching the circuit assembly through the housing.

2. The vital-signs monitor patch of claim 1 wherein the sensor is configured to measure at least one vital sign of the set of body temperature, cardiac pulse rate, respiration rate, blood pressure, and oxygen saturation.

3. The vital-signs monitor patch of claim 2 wherein the sensor comprises one or more electrodes; and wherein the vital-signs monitor patch is configured such that at least a portion of the vital-signs monitor patch is adhered to the skin of the patient when worn, and wherein the electrodes are in electrical contact with the patient's skin when the vital-signs monitor patch is adhered to the patient.

4. The vital-signs monitor patch of claim 3 wherein the electrodes of the patch are connectors to which separate electrodes can be attached, wherein the separate electrodes are in electrical contact with the patient's skin when the vital-signs monitor patch is adhered to the patient.

5. The vital-signs monitor patch of claim 3 wherein the electrodes are operably connected to the components of the circuit assembly through one or more conductive strips on the surface of a flexible substrate.

6. The vital-signs monitor patch of claim 5 wherein the conductive strips on the flexible substrate are operably connected to the circuit assembly through a segment of conductive adhesive.

7. The vital-signs monitor patch of claim 6 wherein the conductive adhesive is either isotropically or anisotropically conductive.

8. The vital-signs monitor patch of claim 6 wherein the conductive strips on the flexible substrate are held in contact with the conductive adhesive by at least one segment of high-strength adhesive.

9. The vital-signs patch of claim 1 wherein the vital-signs monitor patch comprises a cushion layer between the circuit assembly and the housing.

10. The vital-signs patch of claim 1 wherein the vital-signs patch further comprises:
a battery; and
a removable power isolation strip;
wherein the circuit assembly is configured such that the battery is operably isolated from the rest of the circuit assembly when the power isolation strip is in place and the battery is operably connected to the rest of the circuit assembly when the power isolation strip is removed, and wherein the power isolation strip protrudes through an opening in the housing when it is in place; and wherein the opening closes and seals after the power isolation strip is removed.

11. The vital-signs patch of claim 1 wherein the vital-signs patch further comprises:
a battery; and
an externally accessible connector;
wherein the circuit assembly is configured such that the battery is operably isolated from the rest of the circuit assembly until a mating connector is plugged into the externally accessible connector, whereupon the battery is operably connected to the rest of the circuit assembly.

12. A vital-signs monitor patch comprising:
at least one sensor configured to measure a physiological parameter of a patient;
a circuit assembly containing vital-signs monitoring circuitry that analyzes the sensor measurements to generate vital sign signals; and
a housing configured for wearing by a patient and encapsulating the circuit assembly, the housing comprising a first layer and a second layer opposing the first layer, the first and second layers forming a space there between in which the circuit assembly is encapsulated, and a sealing area on the first and second layers at which the first and second layers are sealed together.

13. The vital-signs monitor patch of claim 12 wherein the sensor is configured to measure at least one vital sign of the set of body temperature, cardiac pulse rate, respiration rate, blood pressure, and oxygen saturation.

14. The vital-signs monitor patch of claim 12 wherein the sensor comprises one or more electrodes; and wherein the vital-signs monitor patch is configured such that at least a portion of the vital-signs monitor patch is adhered to the skin of the patient when worn, and wherein the electrodes are in electrical contact with the patient's skin when the vital-signs monitor patch is adhered to the patient.

15. The vital-signs monitor patch of claim 14 wherein the electrodes of the patch are connectors to which separate electrodes can be attached, wherein the separate electrodes are in electrical contact with the patient's skin when the vital-signs monitor patch is adhered to the patient.

16. The vital-signs monitor patch of claim 14 wherein the electrodes are operably connected to the components of the circuit assembly through one or more conductive strips on the surface of a flexible substrate.

17. The vital-signs monitor patch of claim 16 wherein the conductive strips on the flexible substrate are operably connected to the circuit assembly through a segment of conductive adhesive; wherein the conductive strips on the flexible substrate are held in contact with the conductive adhesive by at least one segment of high-strength adhesive.

18. The vital-signs monitor patch of claim 12 wherein the vital-signs monitor patch comprises a cushion layer between the circuit assembly and the housing.

19. The vital-signs monitor patch of claim 12 wherein the vital-signs monitor patch further comprises:
a battery; and
a removable power isolation strip;
wherein the circuit assembly is configured such that the battery is operably isolated from the rest of the circuit assembly when the power isolation strip is in place and the battery is operably connected to the rest of the circuit assembly when the power isolation strip is removed, and wherein the power isolation strip protrudes through an opening in the housing when it is in place, and wherein the opening closes and seals after the power isolation strip is removed.

20. The vital-signs patch of claim 12 wherein the vital-signs patch further comprises:
a battery; and
an externally accessible connector;
wherein the circuit assembly is configured such that the battery is operably isolated from the rest of the circuit assembly until a mating connector is plugged into the externally accessible connector, whereupon the battery is operably connected to the rest of the circuit assembly.
